(19) United States
(12) Patent Application Publication
(10) Pub. No.: US 2024/0074843 A1
(43) Pub. Date: Mar. 7, 2024

(54) Vascular Stent Connector, Vascular Stent-Graft Employing Same, and Methods of Making Same

(71) Applicant: Michael C. Gianturco, New York, NY (US)

(72) Inventor: Michael C. Gianturco, New York, NY (US)

(21) Appl. No.: 18/242,951

(22) Filed: Sep. 6, 2023

Related U.S. Application Data
(60) Provisional application No. 63/404,312, filed on Sep. 7, 2022.

(51) Int. Cl. A61F 2/07

(52) U.S. Cl. CPC A61F 2/07; A61F 2220/0025

(57) ABSTRACT

A vascular stent connector adapted to flexibly connect two stents in a vascular stent-graft, and a vascular stent-graft employing same. The stent connector includes a plurality of flexible connector elements, each of the connector elements including: a first element leg structured to be secured to a first peak of the first stent in the vascular stent-graft; and a second element leg structured to be secured to a second peak of the second stent in the vascular stent-graft. The stent-graft includes the stent connector as well as a length of vascular graft material and at least a first stent and a second stent supporting the vascular graft material. The stent connector elements may be V-shaped, W-shaped, or the like.

### RELATED APPLICATIONS

Priority is claimed from U.S. Provisional Patent Application No. 63/404,312 filed Sep. 7, 2022, entitled METHODS OF CONNECTING STENTS AND APPARATUSES THEREFOR, the entirety of which is incorporated by reference herein.

### BACKGROUND OF THE INVENTION

### Field of the Invention

The invention is directed to devices and methods for connecting stents together. More specifically, the invention is directed to devices and methods for connecting arterial/vascular stents together end-to-end across a sharp angle, e.g., a sharp suprarenal angle, in arteries such as the aorta for repairing abdominal aortic aneurysms, for example.

### Description of Related Art

An abdominal aortic aneurysm (AAA or “triple A”) is most typically diagnosed in men 65 or older. If left untreated the aneurysm may rupture, usually fatally. There are two ways to treat an abdominal aortic aneurysm.

A graft can be installed with open surgery on the abdomen. A graft can also be installed much less invasively by endovascular repair, or EVAR. This requires that a guide wire, sheath, control wires and ultimately a stent-graft be introduced through a cut in the femoral artery. Both open surgical and EVAR techniques involve installing a graft—a synthetic tube which completely replaces the damaged, ballooning aneurysm tissue with a new, synthetic lumen for blood flow. The invention is intended for EVAR.

The EVAR devices are loosely called AAA stents, but more exactly AAA stent-grafts. In a stent-graft, the graft is typically Dacron or Teflon anchored by, shaped, sealed, and reinforced by stents. Most of the stents are self-expanding upon release from a sheath, though some some may be installed and expanded with a balloon catheter. After installation, the stent-graft becomes the secure new path for blood flow, tunneling through and bypassing the aneurysm. The graft may be bifurcated and the two “pant legs” address aneurysms that often extend into the iliac arteries.

Before installation, the graft and its reinforcing stents are compressed into the sheath. They are delivered through the sheath, up the femoral and iliac arteries and into the abdominal aorta. Upon release from the sheath, the stent-graft expands within the aneurysm and bypasses it.

The expanded stent-graft makes a bridge between healthy aortic tissue above and below the aneurysm. At the ends of this bridge, at the “landing zones,” the graft must form tight seals against the interior of the aorta so that blood cannot leak into the aneurysm and keep it inflating.

Men over 65 often have other conditions that argue against open surgery, and endovascular repair using a stent-graft introduced through a femoral slit has become the favored technique over the years since its commercial introduction in 1994. EVAR is suitable for about 85% of the patients found to have an AAA.

The remaining 15% of the patients may be excluded for various reasons but the main reason is a “hostile neck”. The neck of an aneurysm is a short stretch of healthy tissue in the aorta adjacent to the point where the aneurysm begins to balloon. The neck is considered to extend upstream in the aorta from the edge of the aneurysm toward the heart (i.e., proximally) to the lowest of the two branchings from the aorta of the renal arteries. The neck is sometimes called the proximal landing zone because this is where a sealing ring stent (or other sealing mechanism, such as a pneumatic ring) must be secured. In order to achieve a good seal, there must be excellent apposition of the natural lumen of the aorta and the artificial (Teflon or polyester) skin of the stent-graft.

In patients with a “hostile neck,” the neck of the aneurysm creates problems that interfere with the installation and sealing of the stent-graft. Studies have identified the two most significant problems: 1) a large suprarenal angle and 2) a short neck or proximal landing zone.

According to instructions for use for some stent-grafts, the suprarenal angle should not exceed 60 degrees, but in practice, 45 degrees is probably a better and more realistic upper limit for a graft that is not specifically designated as “conformable.”

The shortest proximal landing zone ever found in the instructions for an FDA-approved stent graft was 7 mm, but the device had to be withdrawn. 15 mm is typical. Gore's conformable stent requires at least 1.0 cm but, for higher angulations, 1.5 to 2 cm may be required.

There is some relationship between suprarenal angulation and neck length, but it is neither precisely quantified nor understood. In general, the longer the neck available, the larger the suprarenal angle that can be tolerated.

In patients with hostile necks, there have been two problems, both involving leaks. The first problem is reintervention in more than 20% of the group. This means that shortly after the installation of the stent graft, the physician goes back in via the femoral and does additional adjustment, re-positioning or repair.

If the seal is poor, blood can leak past the lip of the graft and thence into the aneurysm. This is called a Type 1a Endoleak. To stop the leak the physician may install over the sealing area a cuff including another stent to make a better seal.

The second problem is long term. After 6 to 8 years, the graft may begin leaking.

In general, Type 1a Endoleaks are a fairly common problem. Hostile neck installations seem to produce Type 2 endoleaks—i.e. through the aneurysm into the abdomen—but some observers believe the underlying cause might actually be Type 1a endoleaks.

Extreme angulations greater than or equal to 60 or 90 degrees are clearly a problem, but suprarenal angulations less than 45 degrees may also perturb the formation of a good seal. In cases where the stent-graft device is a straight cylinder, it can re-shape the aorta, straightening out the angulation. If the sealing area is short or the apposition of the graft and the endothelial wall of the lumen is distorted or perturbed as a consequence of straightening the angulation, there may be leaks.

The prior art shows various devices that enable a stent to conform more readily to angulated or tortuous vessels. Coronary stents are prominent examples. These solutions make the stent more flexible at a hinge segment or along the longitudinal axis, but this flexibility typically sacrifices radial strength. Radial strength is a force directed orthogonal to the axis of the vessel. It initially expands the stent and, fully expanded, prevents the collapse of a stent-graft. In some types of AAA stent-grafts, a loss of radial strength could compromise graft sealing and encourage Type 1a endoleaks.

More flexible or conforming stents may successfully accommodate gradual changes in angulation. The body of the Gore Excluder Conformable AAA (See FIG. 6) includes multiple expandable ring stents arrayed in distinct tiers and enclosed in folded fabric. The stents are free to move somewhat relative to each other. In effect, the succession of distinct sealing strips of the Gore conformable stent-graft can integrate the curves of (thus conform to) an angulated aorta.

Although this structure is capable of conforming to extreme angulations, it does not readily conform to abrupt angles. It also requires a fairly long neck, that is, from 15 mm to 20 mm for higher angulations.

FIG. 7 is a schematic of the Cook Zenith AAA stent graft uninstalled. At the top of the device are two stents: an uncovered suprarenal stent and, immediately below it, embedded in the graft but still visible, the sealing ring stent. The two stents are joined end-to-end within the proximal end of the graft. This permits some flexibility where the axes of the two stents are conjoined.

Close examination of the uncovered suprarenal stent shows tiny barbs that point out and downward. When the suprarenal stent is installed, these barbs sink into the tissue of the aorta. The idea is to keep the stent-graft from migrating distally (downstream, away from the heart) under the pulsatile pressure and great volumes of descending blood. This type of conjoined suprarenal and sealing ring stent straightens out an angulated aorta but is flexible enough and has enough “give” along its longitudinal axis to accomplish this successfully for angles that do not exceed 45 degrees.

Medtronic manufacturers a stent graft shown in FIG. 8. Like Cook, Medtronic uses an uncovered suprarenal stent connected to a sealing ring stent secured within the top of the graft. Both manufacturers secure the uncovered suprarenal stent to the graft at or near the sealing ring stent. The uncovered suprarenal stent is bigger and more robust than the sealing ring stent. It is left uncovered so that it does not impede blood flow to the two renal arteries.

A goal in connecting the two stents is to convey some of the greater radial strength of the bigger upper stent down to the sealing ring stent. The hope is that this borrowed radial strength will make for a more effective seal between the proximal end of the graft and the aorta.

Until and unless a good seal is achieved between the graft and the endothelial wall of the aorta, blood will continue to leak into and further inflate the aneurysm.

Among the major manufacturers, Cook and Medtronic both use the uncovered suprarenal stent with barbs, linked to a sealing ring stent secured within the top of the graft. Both of these manufacturers secure the uncovered suprarenal stent to the graft at or near sealing ring stent.

These two conjoined stents can be expanded sequentially using control lines. For example, the sealing ring stent and thus the sealing end of the graft may be fully expanded in the distal part of the neck of the aneurysm before, as a subsequent step in the installation, the suprarenal uncovered stent is expanded.

In a straight-line aorta the sequential expansion of two conjoined stents presents no problems. But in an angulated aorta, sequential expansion means, in this example, that the body of the stent-graft has been seated in a segment of the aorta with one angle, but the suprarenal stent is expanding into an adjacent segment of the aorta which may be set at a sharply different angle.

The angle between the two aortic segments might be as great as, for example, 45 degrees. Expansion of the uncovered suprarenal stent will tend to force the two conjoined stents into near alignment along their longitudinal axes. As it does so, the expanding stent forcibly straightens out the angulated aorta. In this process, however, it has been observed that the rim of the sealed graft may slightly peel away from the luminal wall of the vessel. This initial separation can ultimately induce a Type 1a endoleak requiring reintervention.

If there were a flexible connector conjoining the two stents, each stent could conform to the native angle of the segment of the aorta in which it is expanded. There would be no perturbation of the ring stent seal upon installation. This measure should eliminate a known source of endoleaks arising upon installation.

It would be desirable to directly link the suprarenal stent and the sealing ring stent in a stent-graft assembly and to provide a far more flexible connection between these two stents, enabling a stent graft to conform to the shape of an aorta with an abrupt suprarenal angle. It would also be desirable to enable stents and stent-graft assemblies to accommodate abrupt changes in the suprarenal angle, improve sealing against Type Ia endoleaks, and in this way reduce the requisite length of the aneurysm's neck.

Flexible connection alone, however, is not sufficient. It is essential to provide flexibility and yet conserve the transfer of radial strength from the upper, uncovered, suprarenal stent down to the sealing ring stent. Floppy hinges or wobbling connectors could defeat the transfer of radial strength from the suprarenal stent to the sealing stent. It is also essential for the connector to freely adapt to alterations in the distance between the two stents. These changes would be encountered in positioning and re-positioning the two stents upon installation, and in the normal longitudinal changes in the aorta itself in response to pulsatile blood pressure changes. Finally, in view of the longitudinal changes in the distance between the two stents, it would be desirable in an assembly with a flexible connector to move the fixation barbs from the uncovered suprarenal stent to the sealing ring stent, which is a fixed mount relative to the aorta. This would resist downstream migration of the conjoined stents.

The problems of suprarenal angulation have been emphasized here, but it would also be desirable to provide a flexible connector suitable for stents that must conform to infrarenal aortic angles, thoracic aortic stents, and other common stents that must conform to abrupt angles in vessels and ducts.

### SUMMARY OF THE INVENTION

It is an object of the invention to provide a stent connector for a stent-graft assembly that enables stents to accommodate abrupt changes in the suprarenal angle when repairing an aneurysm, thereby improving sealing against Type Ia endoleaks and (in principle) reducing the requisite length of the aneurysm's neck.

It is another object of the invention to provide a connector suitable for stents that must conform to infrarenal aortic angles, thoracic aortic stents, and other common stents that must conform to abrupt angles.

It is another object of the invention to directly link two stents in a vascular graft and provide a far more flexible connection between these two stents, enabling a stent graft to conform to the shape of an aorta or other vessel with an abrupt angle.

It is another object of the invention to provide a connector which can instantly accept and adapt to changes in the end-to-end distance between the two stents to be connected. Such changes may be encountered in installation, re-positioning, and routinely and repeatedly as the aorta responds to the pulsatile flow of blood from the heart.

The above and other objects are fulfilled by the invention, which is a vascular stent connector and stent-graft employing same, and preferably an aortic stent connector and stent-graft, and more preferably still an abdominal aortic aneurysm stent-graft connector and stent-graft.

In one embodiment, the invention includes a vascular stent connector adapted to flexibly connect two stents in a vascular stent-graft. The invention includes a plurality of flexible connector elements, each of the connector elements including: a first element leg structured to be secured to a first stent in the vascular stent-graft; and a second element leg structured to be secured to a second stent in the vascular stent-graft. The first element leg is structured to be secured to a first peak of the first stent and the second element leg being structured to be secured to a second peak of the second stent. Optionally, the second element leg is flexibly connected to the first element leg at a vertex, wherein the connector element is substantially V-shaped. Preferably, the first element leg further includes a first flange structured to be attached to the first peak of the first stent, and the second element leg further includes a second flange structured to be attached to the second peak of the second stent.

Optionally, the inventive stent connector includes the same number of the connector elements as there are of the first and second peaks of the first and second stents. Alternatively, the stent connector includes fewer of the connector elements than there are of the first and second peaks of the first and second stents, to thereby increase the vascular angle to which the stent-graft can be conformed to thereby eliminate interference where the first and second stents are closest to each other.

Optionally, the inventive vascular stent connector further includes at least one intermediate leg flexibly connected to the first element leg at a first vertex and to the second element leg at a second vertex. The at least one intermediate leg may further include a third element leg flexibly connected to the first element leg at the first vertex and a fourth element leg flexibly connected to the third element leg at a third vertex, wherein the fourth element leg is flexibly connected to the second element leg at the second vertex, and wherein the connector element is substantially W-shaped.

In another embodiment, the invention is a vascular stent-graft that includes a length of vascular graft material and at least a first stent and a second stent, the first and second stents supporting the length of vascular graft material. A connector connects the first stent to the second stent; the connector includes a plurality of flexible connector elements. Each of the connector elements includes a first element leg structured to be secured to a first peak of the first stent, and a second element leg structured to be secured to a second peak of the second stent. Optionally, for at least one of the connector elements, the second element leg is flexibly connected to the first element leg at a vertex, and the at least one connector element is substantially V-shaped. Optionally, the first element leg further includes a first flange structured to be attached to the first peak of the first stent, and the second element leg further includes a second flange structured to be attached to the second peak of the second stent. Optionally, the stent connector includes the same number of the connector elements as there are the first and second peaks of the first and second stents. Optionally, the stent connector includes fewer of the connector elements than there are the first and second peaks of the first and second stents to thereby increase the vascular angle to which the stent-graft can be conformed to thereby eliminate interference where the first and second stents are closest to each other.

Optionally, the inventive vascular stent-graft further includes at least one intermediate leg flexibly connected to the first element leg at a first vertex and to the second element leg at a second vertex. Optionally, the at least one intermediate leg includes a third element leg flexibly connected to the first element leg at the first vertex, and a fourth element leg flexibly connected to the third element leg at a third vertex, wherein the fourth element leg is flexibly connected to the second element leg at the second vertex, and wherein the connector element is substantially W-shaped.

In another embodiment, the invention includes a non-transitory computer-readable medium encoded with instructions for creating a vascular stent connector element adapted to flexibly connect two stents in a vascular stent-graft, the medium having: instructions for defining a first element leg structured to be secured to a first stent in the vascular stent-graft; and instructions for defining a second element leg structured to be secured to a second stent in the vascular stent-graft, wherein the second element leg is structured to be flexibly connected to the first element leg at a vertex. Optionally, the medium further includes instructions for defining at least one intermediate leg flexibly attached to the first element leg at a first vertex and to the second element leg at a second vertex. Optionally, the medium further includes instructions for creating the stent connector via at least one of laser cutting or additive manufacturing.

In another embodiment, the invention includes a non-transitory computer-readable medium encoded with instructions for creating conformably linked vascular stents for a vascular stent-graft, the medium having: instructions for defining at least a first stent and a second stent, the first and second stents adapted to support a length of vascular graft material; instructions for defining a connector connecting the first stent to the second stent, the connector including a plurality of flexible connector elements, each of the connector elements including: a first element leg secured to a first peak of the first stent; and a second element leg secured to a second peak of the second stent.

Optionally, the medium further includes instructions for defining that the second element leg is flexibly connected to the first element leg at a vertex, wherein each of the connector elements is substantially V-shaped. Optionally, the medium further includes: instructions for defining a first flange on the first element leg secured to the first peak of the first stent, and instructions for defining a second flange on the second element leg secured to the second peak of the second stent. Optionally, the medium further includes instructions that define the stent connector to include the same number of the connector elements as there are the first and second peaks of the first and second stents. Alternatively, the medium further includes instructions that define the stent connector to include fewer of the connector elements than there are the first and second peaks of the first and second stents to thereby eliminate interference where the first and second stents are closest to each other.

Optionally, the medium further includes instructions for defining at least one intermediate leg flexibly connected to the first element leg at a first vertex and to the second element leg at a second vertex. Optionally, the medium further includes instructions for defining that the at least one intermediate leg includes a third element leg flexibly connected to the first element leg at the first vertex and a fourth element leg flexibly connected to the third element leg at a third vertex, wherein the fourth element leg is flexibly connected to the second element leg at the second vertex, and wherein the connector element is substantially W-shaped.

In another embodiment, the invention includes a method of flexibly connecting two vascular stents in a vascular stent-graft comprising the steps of: a) providing a plurality of flexible connector elements, each of the connector elements including a first element leg structured to be secured to a first stent, and a second element leg structured to be secured to a second stent, wherein the second element leg is flexibly connected to the first element leg; b) attaching first ends of each of the first element legs to the first stent; and c) attaching second ends of each of the second element legs to the second stent. The inventive method optionally further comprises the steps of: providing a first flange on the first end of the first element leg and a second flange on the second end of the second element leg for each of the connector elements, wherein the attaching step b) further comprises the step of attaching the first flange to a first peak of the first stent; and wherein the attaching step c) further comprises the step of attaching the second flange to a second peak of the second stent. The attaching steps b) and c) may optionally be performed as welding steps. The attaching steps b) and c) may optionally be performed as laser spot welding steps.

The invention is a flexible stent connector that nevertheless retains the ability to transmit radial strength from one stent to the other to help seal the graft against the lumen of the aorta and support the vascular structure.

In one embodiment, the stent connector includes a plurality of connector elements. Each connector element includes a first element leg structured to be secured to a first stent, and a second element leg structured to be secured to a second stent, wherein the second element leg is flexibly attached to the first element leg at a vertex, the connector element taking a general V-shape. The first element leg may include a first flange structured to be attached to a peak of the first stent, and the second element leg may include a second flange structured to be attached to a peak of the second stent. The stent connector preferably includes the same number of connector elements as there are peaks of the first and second stents. Typically there are 6 or 8 peaks, but more can be accommodated. A good connection between two 6-peak stents can be made using just 4 hairpin connectors. The deletion of 2 connectors increases the angle to which it can be conformed by eliminating interference where the two stents are closest together.

In general, in securing the inventive connector between two stents, care must be taken to assure that no mechanical interference reduces the length of the arc that can be freely traversed by the connected stents.

In another embodiment, the stent connector includes a plurality of connector elements. Each connector element includes a first element leg structured to be secured to a first stent, and a second element leg structured to be secured to a second stent. A third element leg is flexibly attached to the first element leg at a first vertex, a fourth element leg is flexibly attached to the third element leg at a second vertex, and the fourth element leg is flexibly attached to the second element leg at a third vertex, the connector element taking a general W-shape. The first element leg may include a first flange structured to be attached to a peak of the first stent, and the second element leg may include a second flange structured to be attached to a peak of the second stent. The stent connector preferably includes the same number of connector elements as there are peaks of the first and second stents. As the W connector can double the angle accepted by the V connector, reducing the number of connectors below the number of peaks is not needed.

In another embodiment, the stent connector includes a plurality of connector elements. Each connector element includes a first element leg structured to be secured to a first stent, and a second element leg structured to be secured to a second stent. At least one intermediate leg is flexibly attached to the first element leg at a first vertex and to the second element leg at a second vertex. The first element leg may include a first flange structured to be attached to a peak of the first stent, and the second element leg may include a second flange structured to be attached to a peak of the second stent. The stent connector preferably includes the same number of connector elements as there are peaks of the first and second stents.

The invention also includes a non-transitory computer-readable medium encoded with instructions for creating, e.g., via laser cutting or via additive manufacturing such as 3D printing, a stent connector element, the medium having instructions for defining a first element leg structured to be secured to a first stent, and instructions for defining a second element leg structured to be secured to a second stent, wherein the second element leg is structured to be flexibly attached to the first element leg at a vertex. The invention may also include a non-transitory computer-readable medium encoded with instructions for creating, e.g., via laser cutting or via additive manufacturing such as 3D printing, a stent connector element, the medium having instructions for defining a first element leg structured to be secured to a first stent, instructions for defining a second element leg structured to be secured to a second stent, and instructions for defining at least one intermediate leg flexibly attached to the first element leg at a first vertex and to the second element leg at a second vertex.

The invention further includes a method of making the inventive stent connectors via laser cutting or via additive manufacturing such as 3D printing.

The invention further includes a stent connector itself made via laser cutting or via additive manufacturing such as 3D printing.

The invention further includes a method of attaching two stents in a stent graft assembly by providing a plurality of connector elements, each including a first element leg structured to be secured to a first stent, and a second element leg structured to be secured to a second stent, wherein the second element leg is flexibly attached to the first element leg, either at a vertex or via one or more intermediate legs at two or more vertices; attaching the ends of each of the first element legs to the first stent; and attaching the ends of each of the second element legs to the first stent. The method may further include providing a first flange on the first element leg and a second flange on the second element leg; attaching the first flange to a peak of the first stent; and attaching the second flange to a peak of the second stent. The attaching steps are preferably performed as welding steps and more preferably as laser spot welding steps.

The invention further includes, in view of the longitudinal changes in the distance between the two stents, a shift in the mounting of the fixation barbs from the uncovered suprarenal stent to the sealing ring stent, which is a fixed mount relative to the aorta. This repositioning of the barbs enables them to resist downstream migration of the conjoined stents.

### BRIEF DESCRIPTION OF THE DRAWINGS

FIG. 1A is perspective view of a connector in accordance with a first embodiment of the invention connecting two stents.

FIG. 1B is a perspective view of the connector of FIG. 1A with the suprarenal stent being canted an angle α with respect to the sealing ring stent.

FIG. 1C is a detail view of the barbs, stent, and connector of FIG. 1B.

FIG. 1D is a schematic of the connector and stent-graft of FIG. 1B in situ in an artery.

FIG. 2A is a perspective view of one connector element of the connector of FIGS. 1A-B in accordance with the invention.

FIG. 2B is a detail view of the end of the connector element of FIG. 2A.

FIG. 3A is a top view of the connector element of FIG. 2 in accordance with of the invention.

FIG. 3B is a perspective view of the connector element of FIG. 2 in accordance with of the invention.

FIG. 3C is a front view of the connector element of FIG. 2 in accordance with of the invention.

FIG. 3D is a side view of the connector element of FIG. 2 in accordance with of the invention.

FIG. 4A is a perspective view of one connector element of a connector in accordance with a second embodiment of the invention.

FIG. 4B is perspective view of a connector in accordance with an embodiment of the invention having connector elements as shown in FIG. 4A connecting two stents

FIG. 5A is a top view of the connector element of FIGS. 4A-B in accordance with of the invention.

FIG. 5B is a perspective view of the connector element of FIGS. 4A-B in accordance with of the invention.

FIG. 5C is a front view of the connector element of FIGS. 4A-B in accordance with of the invention.

FIG. 5D is a side view of the connector element of FIGS. 4A-B in accordance with of the invention.

FIG. 6 is a schematic of a GORE® EXCLUDER® conformable AAA stent-graft manufactured by W.L. Gore & Associates, Inc.

FIG. 7 is a schematic of a Cook Medical Zenith Flex® AAA Endovascular Graft.

FIG. 8 is a schematic of a Medtronic ENDUIRANT® II/IIS AAA stent-graft assembly.

FIG. 9 is a schematic of a setup for CNC laser cutting a nitinol semi-cylinder to produce multiple connector elements for stents of a specified diameter.

FIG. 10A is a perspective view of a one-piece stent-connector-stent stent-graft frame of conformably linked stents in accordance with an embodiment of the invention.

FIG. 10B is a detail view of the connector-stent interface of the frame of FIG. 10A.

FIG. 11A is a perspective view of a one-piece multi-stent stent-graft frame in accordance with an embodiment of the invention.

FIG. 11B is a top view of the multi-stent stent-graft frame of FIG. 11A.

### DETAILED DESCRIPTION OF THE INVENTION AND DRAWINGS

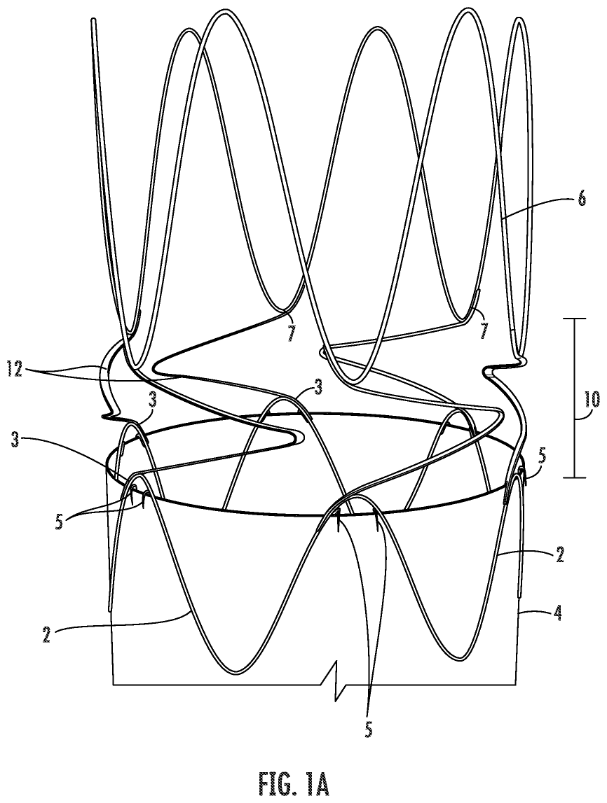

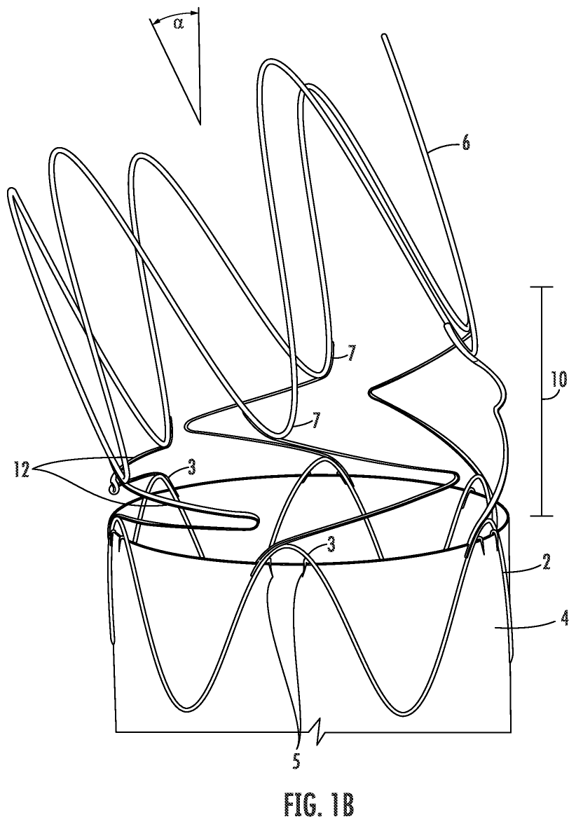

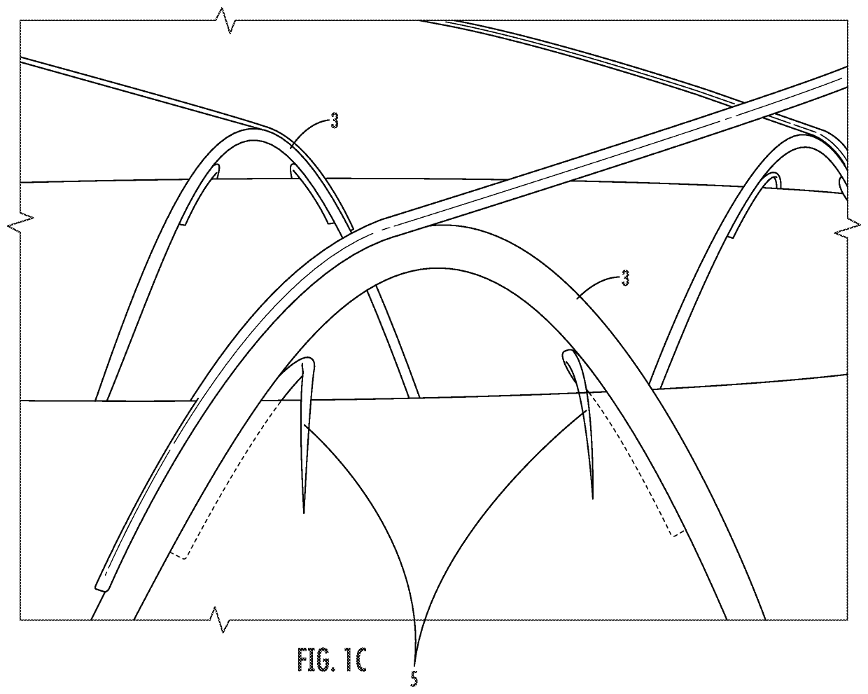

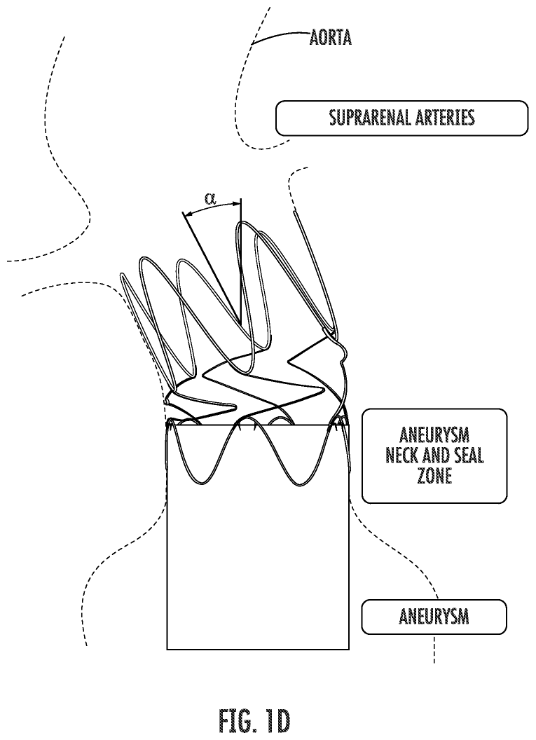

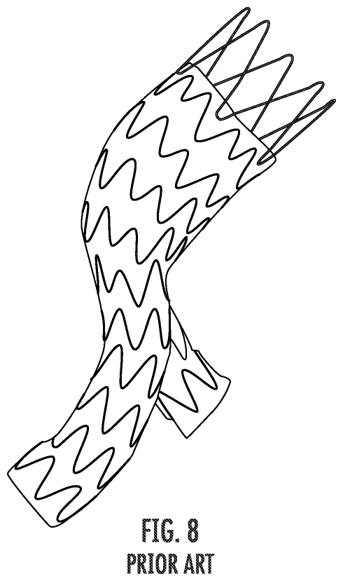

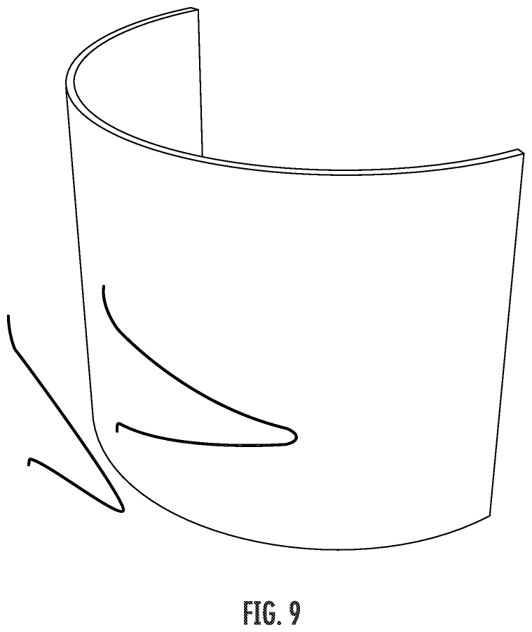

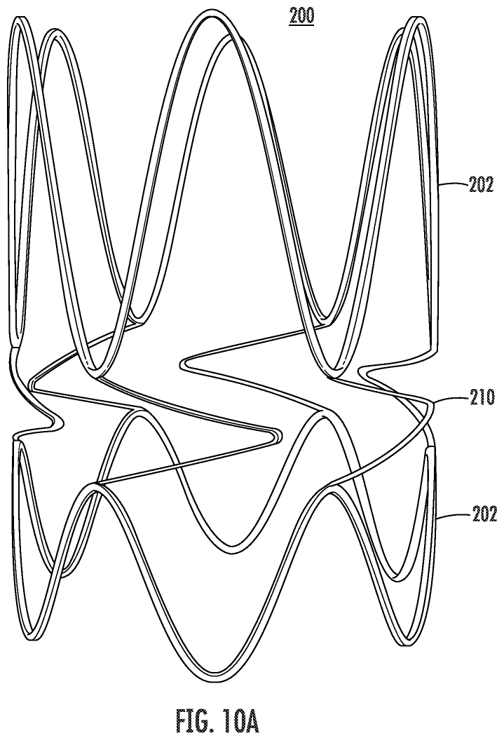

Description will now be given with reference to the attached FIGS. 1-11. It should be understood that these figures are exemplary in nature and in no way serve to limit the scope of the invention, which is defined by the claims appearing hereinbelow.

One embodiment of the invention is depicted in FIGS. 1-3. As shown in FIGS. 1A-D, the inventive connector 10 is contemplated for use with two stents 2 and 6. Stent 2 is covered by a vascular (e.g., arterial) graft 4. As with several conventional AAA stent graft systems, sealing ring stent 2 is embedded in or otherwise covered by graft 4. An uncovered suprarenal stent 6, immediately adjacent to sealing ring stent 2, ascends proximally in FIGS. 1A-B. In the case of a typical AAA stent graft system, suprarenal stent 6 ascends to the level of the aorta where the renal arteries branch. Suprarenal stent 6 is left uncovered by graft 4 to avoid interference with the flow of blood, e.g., to the kidneys. Barbs 5 (best shown in FIG. 1C) are typically mounted thereon to enable stent 6 to lodge firmly in the sidewall of the vessel to prevent/resist downstream migration of the stent-graft assembly. For stents connected by the inventive device, it is necessary to mount barbs 5 on the sealing ring stent rather than the suprarenal stent.

As noted above, stents 2 and 6 are conventionally linked together end-on fairly tightly, resulting in a unified tubular frame joined by a rather inflexible connection. In a straight aorta, this is not a problem. However, in an aorta with a suprarenal angle, the inflexible link between stents is potentially disastrous, as it can actually result in a failure of the blood-tight seal on one side of the aorta between the graft and the aorta. This failed seal can produce an endoleak and potentially reinflate the aneurysm.

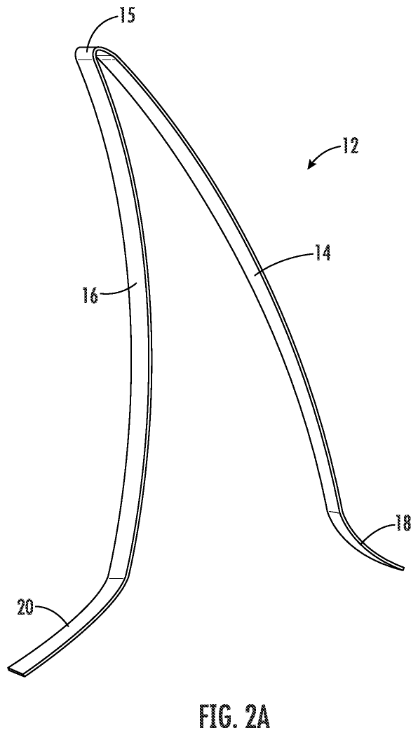

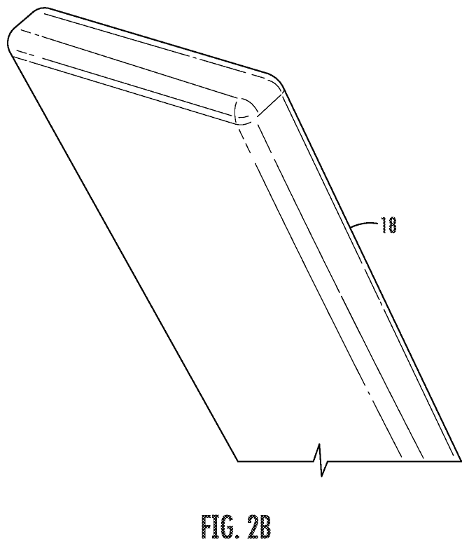

As shown in FIGS. 1A-D, stent connector 10 is provided which enables the overall stent graft assembly to flex at an angle to accommodate an aorta with a suprarenal angle (or other vessel with a bend or turn therein). Connector 10 includes a plurality of connector elements 12 each, in this embodiment, having a general V-shape. As shown in FIG. 2, each connector element 12 includes two legs 14 and 16 attached at a common preferably curved vertex 15. At the free end of leg 14 is preferably provided flange 18, and at the free end of leg 16 is preferably provided flange 20. Flanges 18 and 20 are the portions of connector element 12 that will be secured to stents 2 and 6 at their respective peaks 3 and 7 (see FIGS. 1A-B). The preferred material out of which connector elements 12 are to be made is Nitinol, known for its flexibility, shape memory, and biocompatibility. Other materials may be used in special cases, such as stainless steel.

As noted above, the benefits of connector 10 are manifest in a vascular environment that is not dead straight, e.g., an aorta with a suprarenal angle or bend. FIG. 1A depicts the stent assembly in a straight configuration, while FIG. 1B depicts the stent assembly in an angled configuration with the longitudinal axis of suprarenal stent 6 canted at an angle α with respect to the longitudinal axis of ring stent 2. FIG. 1C is a close up of the barbs mounted on the ring stent, which are tiny. FIG. 1D is a schematic that shows the bent stent as installed in a mildly angulated aorta.

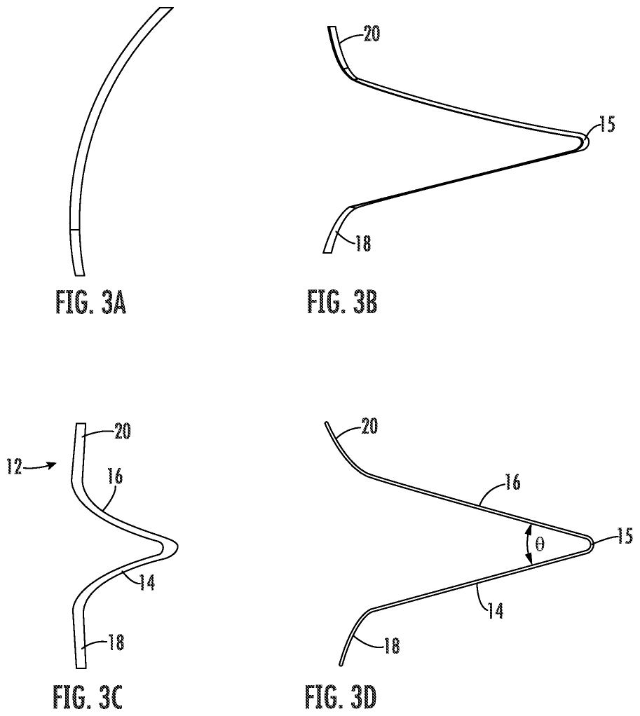

FIG. 2A depicts connector element 12 in perspective. FIG. 2B depicts a close up of the filleting of the end of the connector element. The Nitinol connector is filleted to avoid sharp edges and points. Filleting can be accomplished using ablation techniques including particulate or laser ablation. (It would be easier to fillet steel stents via, e.g., simple machining.) FIG. 3A-D shows connector element 12 in top, perspective, front, and side views, respectively. The side and top views emphasize that element 12 is thin and flexible in its flexing plane but thick in the plane which resists the loss of radial strength transferred from the suprarenal stent to the sealing ring stent.

Note that the curvature and length of the connector arms 14 and 16 will be manufactured to reflect the diameter and curvature specified for a given stent. For AAA stent grafts, these dimensions will change as a function of the diameter of the stent-graft assembled for the aortas of specific patients, that is, a diameter range from roughly 18 mm to 32 mm.

In the illustration, flange 20 is longer than flange 18 because of the respective arc lengths of the peaks of the two exemplary stents. In other applications, the flanges' relative lengths could be reversed, or they could be of equal size.

Flanges 18 and 20 of the individual connector elements 12 are attached to each of the two stents 2 and 6 (see FIG. 1C) at their respective peaks 3 and 7, using, for example, careful spot welding with lasers. It is desired to avoid heat treating the joint, since Nitinol (the preferred material for connector elements 12) forgets its shape memory and may become very flexible at body temperature with too much heat. Other methods of attachment are also contemplated such as purely mechanical fixation, as are other materials to be used for the connector elements, such as stainless steel.

It is envisioned that by using connector 10 having connector elements 12 as outlined above, the resulting system of conjoined stents can conform more readily to abrupt but modest vascular bends such as 25 degrees, while transmitting radial strength from one stent to the other to help maintain a good seal. To put this in context, note that in a population of patients who did not exhibit extreme suprarenal angulation, the median suprarenal angle was found to be 29 degrees. The use of the connector should not affect the requirements or instructions regarding the length of the neck of the aneurysm.

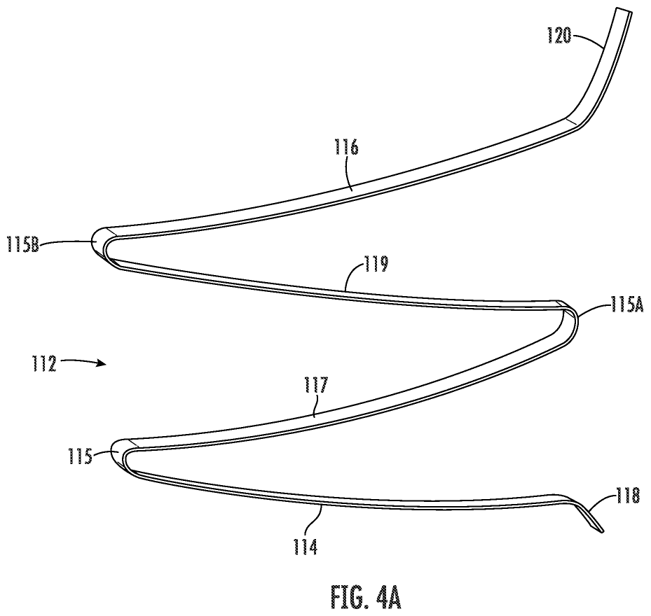

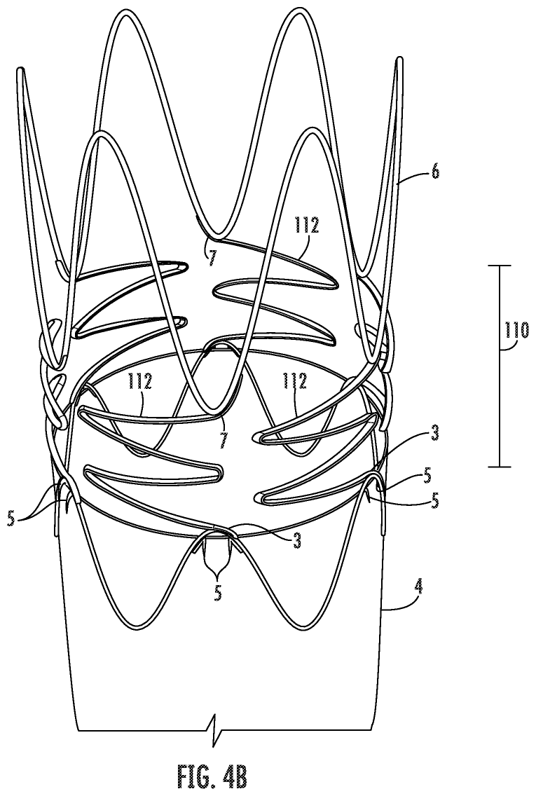

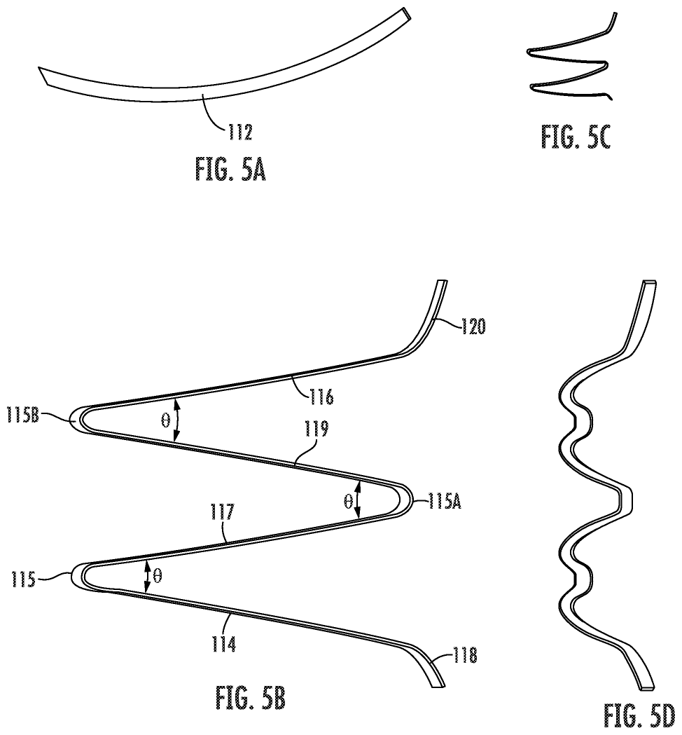

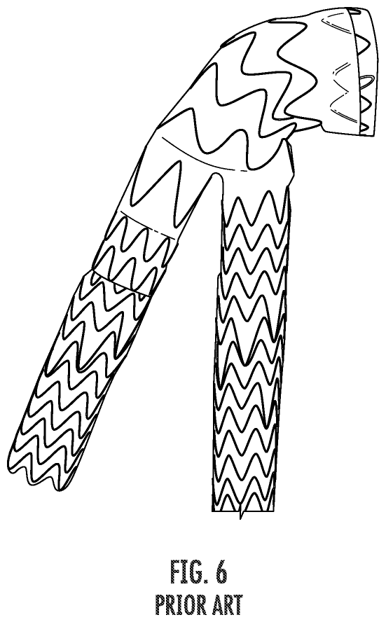

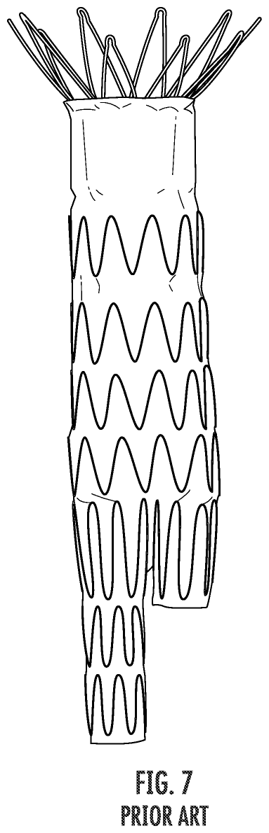

A second embodiment of the inventive stent connector, in this case connector 110, is depicted in FIGS. 4 and 5. It essentially doubles the angle to which the conjoined stents can conform to, in this example, about 50 degrees.

As shown in FIGS. 4 and 5, stent connector 110 (see FIG. 4B) is provided which enables the overall stent-graft assembly to flex at an angle to accommodate an aorta with a suprarenal angle (or other vessel with a bend or turn in it). Connector 110 includes a plurality of connector elements 112 each having a general W-shape (see FIGS. 4A and 5A-D). Each connector element 112 includes two outer legs 114 and 116 and two inner legs 117 and 119. Legs 114 and 117 are attached at a common preferably curved vertex 115. Inner legs 117 and 119 are attached at a common preferably curved vertex 115A. Inner leg 119 is attached to outer leg 116 at a common preferably curved vertex 115B.

At the free end of leg 114 is provided flange 118, and at the free end of leg 116 is provided flange 120. Flanges 118 and 120 are the portions of connector element 112 that will be secured to stents 2 and 6 at their respective peaks 3 and 7 (see FIG. 4B). The preferred material out of which connector elements 112 are to be made is Nitinol, known for its flexibility, shape memory, and biocompatibility. Other materials may be used, such as stainless steel.

Because connector elements 112 have not one but three flexible vertices 115, 115A, and 115B, connector 110 is able to accommodate or conform to a suprarenal aortic angle of up to about 50 degrees. This additional angular range is achieved as a tradeoff of some reduction in the transfer of radial strength from the suprarenal stent to the sealing ring stent. The effect could be mitigated by increasing the thickness of the connector elements in the plane of radial strength.

The curvature of the connector is imparted to it by the anatomical curvature of the aorta. The shape may vary somewhat in response to pulsations in aortic blood flow, so the flexibility of the connector in the plane of flexure is advantageous.

In the illustration, flange 120 is longer than flange 118 because of the respective arc lengths of the peaks of the two exemplary stents. In other applications, the flanges' relative lengths could be reversed, or they could be of equal size.

Flanges 118 and 120 of the individual connector elements 112 are attached to each of the two stents 2 and 6 at their respective peaks 3 and 7, using, for example, careful spot welding with lasers. It is desired to avoid heat treating the joint, since Nitinol (the preferred material for connector elements 12) forgets its shape memory and may become very flexible at body temperature with too much heat. Other methods of attachment are also contemplated, such as purely mechanical fixation, as are other materials to be used for the connector elements.

It is anticipated that by using connector 110 having connector elements 112 as outlined above, the resulting stent graft system can accommodate vascular bends such as a suprarenal aortic angle of, for example, 50 degrees while transmitting radial strength from one stent to the other. To put this in context, note that in a population of patients who did not exhibit extreme suprarenal angulation, the median suprarenal angle was found to be 29 degrees. The use of the connector should not affect the requirements or instructions regarding the length of the neck of the aneurysm.

In view of the longitudinal changes in the distance between the two stents made possible by the connector, it is recommended to shift the mounting of any fixation barbs from the uncovered suprarenal stent to the sealing ring stent, which is fixed once expanded in the aorta. This suggested repositioning of the barbs enables them to resist downstream migration of the conjoined stents.

FIG. 9 depicts one possible method of manufacturing the inventive connector elements: a laser manufacturing technique using Computer Numerical Control (CNC) and semi-cylinders of Nitinol.

The laser cutting beam is projected against a semi-cylinder of nitinol metal. FIG. 9 shows in the foreground the tracking pattern to be followed by the cutting beam. On the surface of the semi-cylinder is projected the cuts to be made by the beam through the curved surface. The thickness of the semi-cylinder defines the thickness of the connector.

In the AAA example of the invention, the outside diameter of the semi-cylinder can be chosen to match the diameter of the aorta for a particular patient, or chosen from a selection of semi-cylinders with typically specified AAA stent and aortic diameters such as 1.8 cm, 2 cm, 4 cm, 6 cm, 8 cm, etc., up to 32 cm.

The software controlling the laser position and the movement of the semi-cylinder can be made to scale the connector (V, W, or other) larger or smaller as appropriate to the diameter of the patient's aorta and thus, to the sizes of the stents to be connected.

One cut will create a single V or W (or other) connector. The semi-cylinder can be mounted on a turntable under computer control. This enables fine rotation coordinated with the laser height controller required to create the cut pattern for a single connector. In addition, the semi-cylinder feedstock can be rotated in abrupt steps to start and create the 4, 6, 8, or more Vs or Ws (or other) required to produce enough connectors to conjoin two stents of specified sizes.

Measures can be taken to keep the feedstock material cool to minimize the heat treating effect and avoid excessive flexibility of the finished nitinol V or W (or other) when it is ultimately exposed to body temperature.

One such measure is to rotate the semi-cylinder from cut to cut in programmed hops from an initial cut to, say, a fresh cut to be made a substantial distance around the arc of the cylinder. Once the site of the initial connector cut has cooled, the programmed laser can hop back to cut a connector from nitinol adjacent to the first cut. A similar approach would be to hop the laser beam from a V (or W, or other) segment to a remote segment of another V (or W, or other), subsequently returning it to cut another segment of the first V (or W, or other) after cooling has occurred.

Conventional methods such as heat sinks and coolant application can also be used to avoid heat treatment of the nitinol. It is important to avoid the floppy hinge or wobbling structure problems that may arise at body temperature with nitinol devices which have been excessively heat treated. Nitinol can become too flexible and lose shape memory. The idea is to maintain flexibility as a generalized property of the connector element, rather than produce a localized flexibility as found, for example, in a heat treated nitinol hinge.

A manufacturing method that integrates stents and connectors into a single piece of Nitinol is also disclosed. The idea is to use as feedstock a Nitinol cylinder the diameter and length of the finished stent. These dimensions would be prescribed for each patient from his or her unique data. The Nitinol cylinder is mounted on a mandrel and rotated and shifted under numerical control as a stationary laser carves out the entire structure of the assembled stent. Some advantages include the ability to quickly custom make a stent that is precisely created for each patient; no jigging, minimal spot welding (for the barbs, in AAA stents), no kits of parts, plus quick delivery. Note that the laser carved stents have rounded rectangular wires. In the AAA embodiment, the larger sealing surface area of the flat exterior surface of the sealing ring stent, relative to the surface of the cylindrical wire stent, can produce a superior seal—a further protection against Type 1a endoleaks.

The one-piece stent approach is particularly appealing for aneurysms affecting the ascending aorta, the great arch, and the descending aorta, as well as the abdominal aorta. The heart produces strong blood pulsations in the upper aorta. The insertion of multiple Vs can enable the stent not only to bend but also to expand and contract along its longitudinal axis, more easily accommodating the energetic passage of blood pulsations. The favored embodiment, however, is the abdominal aortic (AAA) stent.

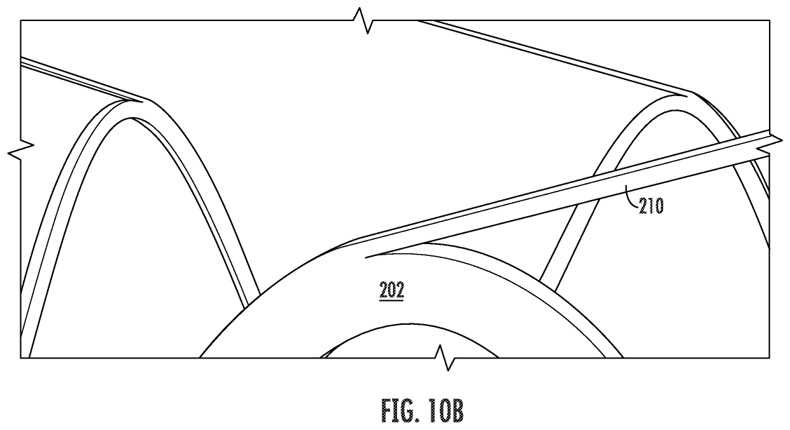

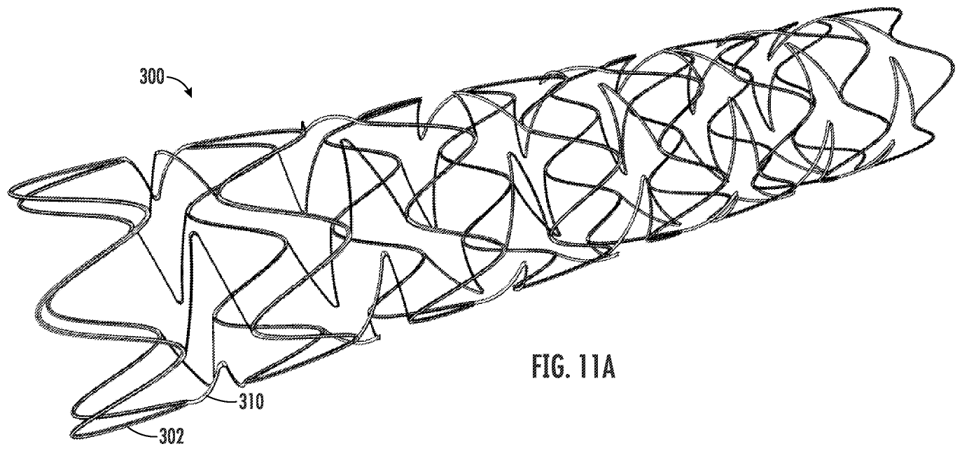

FIG. 10A depicts a one-piece stent-connector-stent stent-graft frame 200 of conformably linked stents. From top to bottom, frame 200 includes a suprarenal stent 202, an inventive V-connector 210, and a sealing ring stent 202. A length of graft material (not shown, e.g., PTFE or Dacron) is incorporated with and supported by frame 200. FIG. 10B is a close up of the connection point between a V-connector 210 and a peak of a sinusoidal sealing ring stent 202. Note that the V-connector is integral with the stents. The entire structure is one piece.

The one piece stent-connector-stent assembly shown in FIGS. 10A-B is optionally made by NC laser cutting a Nitinol cylinder rotating and shifting under computer control. The cylinder contains a mandrel to restrict laser cutting to the part the cylinder exposed to the incoming beam. Above, the attachment of the V- (and W-) connectors was described by spot welding of flanges to the Nitinol stents. This would necessarily require precise jigging of individual V- or W-connectors. Since aortic stents may be manufactured in as many as 14 different diameters, precise jigging and careful spot welding (to avoid heat treating) could become a considerable challenge.

Manufacturing as one single integral piece a connector and two stents, or multiple connectors and multiple stents, eliminates the steps of jigging and spot welding connectors. The AAA stent subassembly/frame 200 shown in FIG. 10A is preferably manufactured as one piece under numerical computer control. This enables easier bespoke manufacturing of stents and stent components with diameters, lengths and angulations (i.e., V- or W-positioning) designed from CT scan data to precisely meet the design requirements for individual patients. The assembly shown in FIG. 10A is freshly laser cut. Subsequent manufacturing steps include laser ablation or particle ablation to fillet sharp edges, and polishing e.g., electropolishing, so that no sharp edges are presented to the endothelium of the aorta.

Note that at the hinge points where the V-connectors join to the suprarenal stent and to the sealing ring stent, the filleting radius is much reduced. Too pronounced a fillet could stiffen these joints. For example, the radius of the fillet near the hinge points can be an order of magnitude smaller than the radius of the generous fillets along the edges of the stents. The fillet radiuses of the Vs' edges are also kept modest to conserve flexibility.

The AAA stent subassemblies shown in FIG. 10A-B are manufactured as one piece under numerical computer control. This enables easier and more rapid bespoke manufacturing of stents and stent components with diameters, lengths and angulations (i.e., V or W positioning) designed from CT scan data to precisely meet the requirements of individual patients.

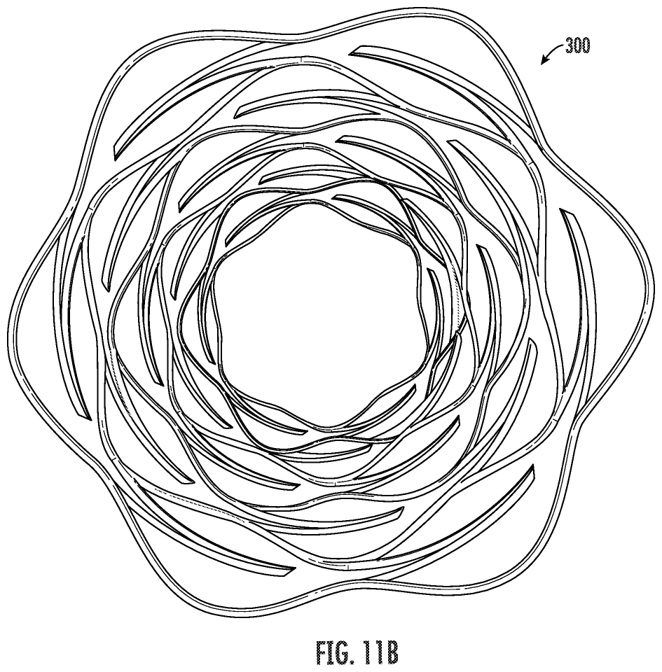

FIG. 11A is a perspective view of a one-piece multi-stent stent-graft frame 300 in accordance with an embodiment of the invention. Frame 300 is a long stent graft frame manufactured by laser cutting—in one piece. Sinusoidal stents 302 alternate with connectors 310, here utilizing V-connector elements (although W- or other shaped connector elements may be employed as desired/needed).

Note that the V- and W-connectors provide two kinds of flexibility. The more evident flexibility enables the stent to “turn corners” rather abruptly at angulations in the aorta. This would of course also be helpful in conforming a stent to the curve of the great arch.

The second type of flexibility inherent in the connector is springiness along the longitudinal axis of the stent. This could be of particular importance in the upper aorta. It has been observed that some conventional aortic stents tend to stiffen the aorta and limit its ability to flex normally in response to oncoming impulses of blood as the heart beats. This effect may have consequences in refracting pressure impulses back to the heart. An aortic stent constructed with the inventive V- or W-connectors has the ability to help absorb incoming pressure waves by flexing along the stent's longitudinal axis.

FIG. 11B is a top view of stent-graft frame 300.

The invention includes four components—a plain or suprarenal stent, a V-connector, a W-connector, and a sealing ring stent. Each of these can be specified in different diameters and lengths and thicknesses.

The components can be combined in different ways, top to bottom. A favored AAA embodiment consists of a suprarenal stent on top, a V- or W-connector in the middle, and a sealing ring stent on the bottom.

A stent intended for the great arch, however, could be made by alternating between plain (straight line) stents and V-connectors and repeating this one-two sequence for the length of the great arch.

A stent for an angulated descending aorta could require long straight sequences interrupted at measured intervals by V-connectors or W-connectors at kinks, and followed by a resumption of straight line stents.

Prescription software is contemplated, one entry page for each type of aortic stent, with entry fields for fully expanded stent diameters, lengths of components, total length, sequences of components, sites of angulation, etc. The form could be filled out with the help of the patient's CT scan displayed on the screen.

The invention is not limited to the above description. For example, although there are described two-leg and four-leg connector elements, other numbers of legs (e.g., 3, 5, etc.) may be provided if the corresponding stiffness of the overall element is adjusted accordingly.

Additionally, a single stent-graft can employ a mix of V-connector elements, W-connector elements, and/or other connectors. Meaning, a single stent-graft frame may include a first connector having all V-connector elements and a second connector having all W-connector elements (or other elements), and so on. Alternatively, a single connector in accordance with an embodiment of the invention may include a mix of different connector elements (V-, W-, other) within the same connector ring. An aorta (or other vessel) may have more than one angle. For smaller angles up to ˜25 degrees, one might specify a V-connector. For larger angles from 25 to ˜50 degrees one might specify a W-connector. Multiple angles in different planes are common, for example, in the descending aorta. Eliminating one or more connector elements is a way to eliminate a site of mechanical interference where the two stents are closest together. It can thus add somewhat to the angles which can be freely achieved between the stents.

Also, above are described inventive stent connectors and inventive stent connection methods. However, the invention also includes a non-transitory computer-readable medium encoded with instructions for creating, e.g., via additive manufacturing such as 3D printing, a stent connector element, the medium having instructions for defining a first element leg structured to be secured to a first stent, instructions for defining a second element leg structured to be secured to a second stent, wherein the second element leg is structured to be flexibly attached to the first element leg at a vertex. The invention further includes a method of making the inventive stent connector via additive manufacturing such as 3D printing. The invention further includes a stent connector itself made via additive manufacturing such as 3D printing.

It should be understood that, in the context of this disclosure, “at least one of” followed by a series of elements means any one of the elements in the series or any combination of the elements in the series, including all of the elements. So, for example, a recitation of “at least one of A, B, or C” means any of A, B, C, A+B, A+C, B+C, or A+B+C.

Having described certain embodiments of the invention, it should be understood that the invention is not limited to the above description or the attached exemplary drawings. Rather, the scope of the invention is defined by the claims appearing hereinbelow and includes any equivalents thereof as would be appreciated by one of ordinary skill in the art.

What is claimed is:

1. A vascular stent connector, adapted to flexibly connect two stents in a vascular stent-graft, comprising:
a plurality of flexible connector elements, each of said connector elements including:
a first element leg structured to be secured to a first stent in the vascular stent-graft; and
a second element leg structured to be secured to a second stent in the vascular stent-graft,
said first element leg being structured to be secured to a first peak of the first stent and said second element leg being structured to be secured to a second peak of the second stent.

2. A vascular stent connector according to claim 1, wherein said second element leg is flexibly connected to said first element leg at a vertex, and wherein said connector element is substantially V-shaped.

3. A vascular stent connector according to claim 1, said first element leg further comprising a first flange structured to be attached to the first peak of the first stent, and said second element leg further comprising a second flange structured to be attached to the second peak of the second stent.

4. A vascular stent connector according to claim 1, wherein said stent connector includes the same number of said connector elements as there are of the first and second peaks of the first and second stents.

5. A vascular stent connector according to claim 1, wherein said stent connector includes fewer of said connector elements than there are of the first and second peaks of the first and second stents to thereby eliminate interference where the first and second stents are closest to each other.

6. A vascular stent connector according to claim 1, further comprising at least one intermediate leg flexibly connected to said first element leg at a first vertex and to said second element leg at a second vertex.

7. A vascular stent connector according to claim 6, said at least one intermediate leg comprising:
a third element leg flexibly connected to said first element leg at said first vertex; and
a fourth element leg flexibly connected to said third element leg at a third vertex,
wherein said fourth element leg is flexibly connected to the second element leg at said second vertex, and
wherein said connector element is substantially W-shaped.

8. A vascular stent-graft, comprising:
a length of vascular graft material;
at least a first stent and a second stent, said first and second stents supporting said length of vascular graft material;
a connector connecting said first stent to said second stent, said connector comprising a plurality of flexible connector elements, each of said connector elements including:
a first element leg structured to be secured to a first peak of said first stent; and
a second element leg structured to be secured to a second peak of said second stent.

9. A vascular stent-graft according to claim 8, wherein for at least one of said connector elements said second element leg is flexibly connected to said first element leg at a vertex, and wherein said at least one connector element is substantially V-shaped.

10. A vascular stent-graft according to claim 8, said first element leg further comprising a first flange structured to be attached to said first peak of said first stent, and said second element leg further comprising a second flange structured to be attached to said second peak of said second stent.

11. A vascular stent-graft according to claim 8, wherein said stent connector includes the same number of said connector elements as there are said first and second peaks of said first and second stents.

12. A vascular stent-graft according to claim 8, wherein said stent connector includes fewer of said connector elements than there are said first and second peaks of said first and second stents to thereby eliminate interference where said first and second stents are closest to each other.

13. A vascular stent-graft according to claim 8, further comprising at least one intermediate leg flexibly connected to said first element leg at a first vertex and to said second element leg at a second vertex.

14. A vascular stent-graft according to claim 13, said at least one intermediate leg comprising:
a third element leg flexibly connected to said first element leg at said first vertex; and
a fourth element leg flexibly connected to said third element leg at a third vertex,
wherein said fourth element leg is flexibly connected to the second element leg at said second vertex, and
wherein said connector element is substantially W-shaped.

15. A non-transitory computer-readable medium encoded with instructions for creating a vascular stent connector element adapted to flexibly connect two stents in a vascular stent-graft, the medium having:
instructions for defining a first element leg structured to be secured to a first stent in the vascular stent-graft; and
instructions for defining a second element leg structured to be secured to a second stent in the vascular stent-graft,
wherein the second element leg is structured to be flexibly connected to the first element leg at a vertex.

16. A non-transitory computer-readable medium encoded with instructions for creating a vascular stent connector element adapted to flexibly connect two stents in a vascular stent-graft in accordance with claim 15, the medium further having instructions for defining at least one intermediate leg flexibly attached to the first element leg at a first vertex and to the second element leg at a second vertex.

17. A non-transitory computer-readable medium encoded with instructions for creating a vascular stent connector element adapted to flexibly connect two stents in a vascular stent-graft in accordance with claim 15, the medium further having instructions for creating the stent connector via at least one of laser cutting or additive manufacturing.

18. A non-transitory computer-readable medium encoded with instructions for creating conformably linked vascular stents for a vascular stent-graft, the medium having:
instructions for defining at least a first stent and a second stent, the first and second stents adapted to support a length of vascular graft material;
instructions for defining a connector connecting the first stent to the second stent, the connector including a plurality of flexible connector elements, each of the connector elements including:
a first element leg secured to a first peak of the first stent; and
a second element leg secured to a second peak of the second stent.

19. A non-transitory computer-readable medium encoded with instructions for creating conformably linked vascular stents for a vascular stent-graft according to claim 18, the medium further having instructions for defining that the second element leg is flexibly connected to the first element leg at a vertex, wherein each of the connector elements is substantially V-shaped.

20. A non-transitory computer-readable medium encoded with instructions for creating conformably linked vascular stents for a vascular stent-graft according to claim 18, the medium further having:
instructions for defining a first flange on the first element leg secured to the first peak of the first stent, and
instructions for defining a second flange on the second element leg secured to the second peak of the second stent.

21. A non-transitory computer-readable medium encoded with instructions for creating conformably linked vascular stents for a vascular stent-graft according to claim 18, wherein the medium further has instructions that define the stent connector to include the same number of the connector elements as there are the first and second peaks of the first and second stents.

22. A non-transitory computer-readable medium encoded with instructions for creating conformably linked vascular stents for a vascular stent-graft according to claim 18, wherein the medium further has instructions that define the stent connector to include fewer of the connector elements than there are the first and second peaks of the first and second stents to thereby eliminate interference where the first and second stents are closest to each other.

23. A non-transitory computer-readable medium encoded with instructions for creating conformably linked vascular stents for a vascular stent-graft according to claim 18, the medium further having instructions for defining at least one intermediate leg flexibly connected to the first element leg at a first vertex and to the second element leg at a second vertex.

24. A non-transitory computer-readable medium encoded with instructions for creating conformably linked vascular stents for a vascular stent-graft according to claim 23, the medium further having instructions for defining that the at least one intermediate leg includes a third element leg flexibly connected to the first element leg at the first vertex and a fourth element leg flexibly connected to the third element leg at a third vertex, wherein the fourth element leg is flexibly connected to the second element leg at the second vertex, and wherein the connector element is substantially W-shaped.
